(19) United States
(12) Patent Application Publication
(10) Pub. No.: US 2026/0123980 A1
(43) Pub. Date: May 7, 2026

(54) ELECTROSURGICAL INSTRUMENT AND ELECTROSURGICAL APPARATUS

(71) Applicant: Creo Medical Limited, Chepstow, Gwent (GB)

(72) Inventors: Louis TURNER, Chepstow (GB); Steven THOMAS, Chepstow (GB); Warren JONES, Chepstow (GB); Christopher Paul HANCOCK, Chepstow (GB); George Christian ULLRICH, Chepstow (GB); Duncan James Foster FITZSIMONS, St. Albans (GB)

(21) Appl. No.: 19/114,347

(22) PCT Filed: Aug. 4, 2023
(86) PCT No.: PCT/EP2023/071646

§ 371 Date: Mar. 21, 2025

(30) Foreign Application Priority Data
Sep. 23, 2022 (GB) 2213944.8

(51) Int. Cl. A61B 18/14; A61B 18/00; A61B 18/12; A61B 18/18

(52) U.S. Cl. CPC A61B 18/1445; A61B 18/1206; A61B 18/1815; A61B 2018/00029; A61B 2018/00178; A61B 2018/00184; A61B 2018/00601; A61B 2018/0063; A61B 2018/00702; A61B 2018/00732; A61B 2018/00982; A61B 2018/00994; A61B 2018/1455; A61B 2018/1823

(57) ABSTRACT

Various aspects disclose an electrosurgical instrument designed for sealing or cutting tissue. The instrument features an instrument shaft with a transmission line to convey microwave or radiofrequency electromagnetic energy. It includes a first jaw with a first surface and a distal end face, and a second jaw with a second surface, both attached to the shaft. The instrument is equipped with first and second electrodes for emitting microwave or radiofrequency energy. A first isolating portion electrically separates the two electrodes. The jaws can move between an open position, allowing tissue insertion between the surfaces, and a closed position, clamping the tissue. The first and second electrodes are exposed on the first surface and distal end face. In the closed position, the second jaw covers the exposed electrodes on the first surface and distal end face, ensuring effective tissue engagement and energy application for surgical procedures.

### FIELD OF THE INVENTION

The invention relates to an electrosurgical instrument for sealing and/or cutting tissue. The electrosurgical instrument is configured to grasp biological tissue and deliver microwave energy into the grasped tissue to seal the tissue by coagulation or cauterization. The electrosurgical instrument may be used to apply pressure to close one or more blood vessels before applying electromagnetic radiation (preferably microwave energy) to seal the blood vessel(s). The electrosurgical instrument may also be arranged to cut, e.g. separate or divide, the vessel or surrounding tissue before or after coagulation or sealing, e.g. using radiofrequency (RF) energy and/or a mechanical cutting element, such as a blade. The invention may be applied to a vessel sealer for use in laparoscopic surgery or open surgery as well as to an endoscopic instrument.

The invention also relates to an electrosurgical apparatus for sealing and cutting tissue which comprises a generator unit for generating radiofrequency and/or microwave electromagnetic energy, and the electrosurgical instrument.

### BACKGROUND TO THE INVENTION

Electrosurgical instruments for delivering heat energy into grasped biological tissue are known. For example, it is known to deliver microwave energy from a bipolar electrode arrangement in the jaws of a forceps. The microwave energy may be used to seal a vessel by thermal denaturation of extracellular matrix proteins (e.g. collagen) within the vessel wall. The heat energy may also cauterize the grasped tissue and facilitate coagulation.

Such devices typically find application on the end of minimally invasive surgical laparoscopic tools but can equally find use in other clinical procedural areas such as gynecology, endourology, gastrointestinal surgery, ENT procedures, or endoscopic procedures. Depending on the context of use, these devices can have differing physical construction, size, scale and complexity.

For example, a gastrointestinal instrument might be nominally of 3 mm diameter mounted on to the end of a very long flexible shaft. In contrast, a laparoscopic instrument may be used on the end of an industry standard nominal 5 mm or 10 mm diameter rigid or steerable steel shaft.

U.S. Pat. No. 6,585,735 describes an endoscopic bipolar forceps in which the jaws of the forceps are arranged to conduct bipolar energy through the tissue held therebetween.

EP 2 233 098 describes microwave forceps for sealing tissue in which the sealing surfaces of the jaws include one or more microwave antennas for radiating microwave energy into tissue grasped between the jaws of the forceps.

WO 2015/097472 describes electrosurgical forceps in which one or more pairs of non-resonant unbalanced lossy transmission line structures are arranged on the inner surface of a pair of jaws.

### SUMMARY OF THE INVENTION

At its most general, the present invention provides various types of electrosurgical instruments that can enable fine tissue cutting and dissection to be performed on tissue. Moreover, the electrosurgical instruments may provide additional functionality, such as sealing biological tissue, such as (blood) vessels, using a confined microwave field that can yield a well-defined seal location with low thermal margin. With these additional functions, fewer device interchanges may be needed during a procedure.

The electrosurgical instruments disclosed herein may be used in any type of surgical procedure, but it is expected to find particular utility for non-invasive or minimally invasive procedures. For example, the electrosurgical instruments may be configured to be introduced to a treatment site through an instrument channel of a surgical scoping device, such as a laparoscope or an endoscope.

According to a first aspect of the present invention, there is provided an electrosurgical instrument for sealing and/or cutting tissue which comprises an instrument shaft, a first jaw, a second jaw, a first electrode, a second electrode, and a first isolating portion. The instrument shaft comprises a (coaxial) transmission line for conveying microwave electromagnetic energy. The first jaw is attached to the instrument shaft and includes a first surface as well as a distal end face. The second jaw is attached to the instrument shaft and includes a second surface. The first isolating portion electrically isolates the first electrode from the second electrode. The first and/or second electrodes are configured to emit microwave and/or radiofrequency electromagnetic energy. The first jaw and the second jaw can be moved between an open position, in which the tissue can be inserted between the first surface and the second surface, and a closed position, in which the first and second surfaces are brought together to clamp, hold and/or grasp tissue therebetween. The first electrode and the second electrode are exposed on the first surface and on the distal end face. In the closed position, the second jaw covers sections of the first electrode and/or of the second electrodes that are exposed on the first surface and the distal end face.

The first and second electrodes in the pair of jaws can operate to provide one or more (e.g. two) localized seals for a biological vessel/tissue gripped between the first and second jaws. Further, the first electrode and the second electrode can be used for radiofrequency cutting at the distal end face. In the open position of the first and second jaws in which the second jaw does not cover the section of the first electrode and the second electrode exposed on the distal end face, the sections of the first electrode and the second electrode that are exposed on the distal end face can act as an active electrode and a return electrode for radiofrequency cutting. This may allow fine cutting at the distal end face of the electrosurgical instrument, for example, for cutting a hole into tissue so that the electrosurgical instrument can be further advanced into and/or through the tissue, for example, as part of a tunnelling procedure. Further, the first and second electrodes exposed on the distal end face can be used to cut fine or small sections of tissue that are clamped or grasped between the distal end face and the second jaw. Thus, tissue can be “nibbled”.

In addition to the above-described modes of use, the invention may allow for another possible mode of cutting tissue by the mechanically moving a centre cutter (e.g. a blade) instead of radiofrequency energy. The geometry of an antenna (e.g. the first and second electrode) of the first jaw in this configuration may allow a mechanically moving blade (or moving radiofrequency electrode) in the centre channel or groove to move straight forward right up to, or possibly even a little beyond a distal end face of the first jaw, whilst still safely covered over by the second jaw at the distal end face (e.g. an overhang portion). This action might allow, for example, simultaneous radiofrequency cutting or microwave sealing (from the antenna) and mechanical cutting (from the blade) right up to the tip (e.g. a distal end face) between the first jaw and the second jaw. This enables tissue to be sealed and “nibbled” without having to rely only on radiofrequency energy as the means to part tissue.

In use, the electrosurgical instrument may thus perform vessel/tissue sealing and vessel/tissue dividing. Vessel/tissue sealing is typically the application of pressure to squash the walls of a biological vessel together, followed by the application of some form of thermal energy. In the invention, the thermal energy is applied by the first and second electrodes to the gripped tissue using the microwave electromagnetic energy. The pressure to the tissue can be applied by the first and/or second electrodes (e.g. the first and second sealing areas) and/or other parts of the first and second jaws. The applied electromagnetic energy disrupts/denatures the tissue cells and forms an amalgam of collagen predominant in vessel/tissue walls, which effectively bonds the vessel/tissue walls together. With time, post operatively, cellular recovery and regrowth occurs to reinforce the seal further. Vessel/tissue dividing is a process of cutting through a continuous biological vessel/tissue to separate it into two pieces. It is normally performed after a vessel/tissue is first sealed. Vessel/tissue dividing is performed by the cutting device, which is discussed in more detail below. As described above, the vessel/tissue dividing is locally offset from the vessel/tissue sealing. In particular, the vessel/tissue dividing occurs between the vessel/tissue seals. Stated differently, the instrument is configured such that vessel/tissue dividing occurs at a different location to vessel/tissue sealing. Dependent on the separation of the electrodes and the electromagnetic field generated by those, for some of the embodiments described herein, one large seal can be provided, e.g. spanning over the complete first surface. This seal is then cut through. This can be an option due to the mechanical cut and/or controlled and varied pressure for radiofrequency cutting that is possible with this invention.

Herein, the terms “proximal” and “distal” refer to the ends of the electrosurgical instrument, the shaft, and/or the coaxial transmission line further from and closer to a treatment site respectively. Thus, in use the proximal end is closer to a generator unit for providing the RF and/or microwave energy, whereas the distal end is closer to the treatment site, i.e. the patient.

The term “conductive” is used herein to mean electrically conductive, unless the context dictates otherwise.

The term “longitudinal” used below refers to the direction along the instrument channel parallel to the axis of the coaxial transmission line. The term “lateral” refers to a direction that is perpendicular to the longitudinal direction. The term “inner” means radially closer to the centre (e.g. axis) of the instrument channel. The term “outer” means radially further from the centre (axis) of the instrument channel.

The term “electrosurgical” is used in relation to an instrument, apparatus or tool which is used during surgery and which utilizes radiofrequency (RF) electromagnetic (EM) energy and/or microwave EM energy. Herein, RF EM energy may mean a stable fixed frequency in a range 10 kHz to 300 MHZ, preferably in a range from 100 kHz to 5 MHz, and more preferably in a range from 360 to 440 kHz. The microwave EM energy may mean electromagnetic energy having a stable fixed frequency in the range 300 MHz to 100 GHZ. The RF EM energy should have a frequency high enough to prevent the energy from causing nerve stimulation. In use, the magnitude of the RF EM energy and the duration for which it is applied may be selected to prevent the energy from causing tissue blanching or unnecessary thermal margin or damage to the tissue structure. Preferred spot frequencies for the RF EM energy include any one or more of: 100 KHz, 250 KHz, 400 KHz, 500 kHz, 1 MHz, 5 MHz. Preferred spot frequencies for the microwave EM energy include 915 MHz, 2.45 GHZ, 5.8 GHz, 14.5 GHz, 24 GHZ. 2.45 GHZ and/or 5.8 GHz may be preferred.

The microwave electromagnetic energy and the radiofrequency electromagnetic energy may be conveyed along a common signal pathway through the instrument shaft. For example, a coaxial cable may provide the common signal pathway for conveying both the microwave energy and the radiofrequency energy. In this arrangement, the transmission line may comprise an inductive filter for blocking the microwave energy from the cutting element, and a capacitive filter for blocking the radiofrequency energy from the first and second electrodes. In an alternative arrangement, the radiofrequency energy and microwave energy are conveyed along separate pathways within the instrument shaft (the transmission line includes separate pathways), wherein the inductive filter and capacitive filter are provided at a proximal end of the instrument shaft, e.g. in a handle. For example, a coaxial cable is provided for conveying the microwave electromagnetic energy while two or more wires are provided for conveying the radiofrequency electromagnetic energy.

The instrument shaft may be dimensioned to fit within an instrument channel of a surgical scoping device. The surgical scoping device may be a laparoscope or an endoscope. Surgical scoping devices are typically provided with an insertion tube that is a rigid or flexible (e.g. steerable) conduit that is introduced into a patient's body during an invasive procedure. The insertion tube may include the instrument channel and an optical channel (e.g. for transmitting light to illuminate and/or capture images of a treatment site at the distal end of the insertion tube). The instrument channel may have a diameter suitable for receiving invasive surgical tools. The diameter of the instrument channel may be equal to or less than 13 mm, preferably equal to or less than 10 mm, and more preferably, especially for flexible insertion tubes, equal to or less than 5 mm.

The instrument shaft and the transmission line may be flexible so that they can be inserted into the instrument channel of the scoping device. Further, the transmission line may be arranged within a lumen of the shaft. The instrument shaft may cover and/or shield the transmission line. The transmission line may extend from a distal end to a proximal end of the electrosurgical instrument. In particular, the transmission line electrically connects the first electrode and the second electrode to the generator unit.

The electrosurgical instrument discussed herein may find applicability in other tissue welding techniques. For example, the energy delivery structure may be used as an alternative to staples. In some abdominal procedures, staple guns are used to deliver 50 to 100 small staples that are fired simultaneously between jaws that can have a length of 70 mm or more, or from an annular jawed arrangements with diameters of 20 to 50 mm. In this type of application multiple antenna structures such as those discussed herein may be used to cover the required length. The antenna structures may be arranged in any number of array forms to be activated simultaneously, sequentially or progressively in a suitable manner.

The first jaw and/or the second jaw may be movable relative to their instrument shaft. The first jaw and/or the second jaw may be attached to the instrument shaft via a joint or hinge. The joint may include a pivot axis around which the first jaw and/or the second jaw may rotate. The first jaw and/or the second jaw may be activated by one or more actuation rods or control wires respectively connected to the first jaw and/or the second jaw. The one or more actuation rods or control wires may extend within the instrument shaft to a proximal end of the electrosurgical instrument. The one or more actuation rods may be connected to a handle with which the first and/or second jaws can be actuated, e.g. opened and/or closed. The electrosurgical instrument comprises an actuation mechanism which converts a back-and-forth movement of the actuation rod(s) or control wire(s) into a rotational movement of the first jaw and/or the second jaw.

For example, both jaws can be movable, e.g. rotatable around a (common) pivot axle. In another embodiment, one of the jaws is fixed to the shaft and the other jaw is movable relative to the one jaw.

In the open position, the first jaw and the second jaw are (maximally) spaced apart so that there is a free space between the first surface of the first jaw and the second surface of the second jaw. In this way, tissue can be inserted between the first surface and the second surface in the open position. Usually, the first jaw and the second jaw are moved towards the tissue such that the tissue is pushed into the space between the first surface and the second surface in the open position of the first jaw and the second jaw.

By moving the first jaw and/or the second jaw from the open position to the closed position, the tissue between the first surface and the second surface can be grasped and/or clamped between the first surface and the second surface and/or between the distal end face and a surface area of the second jaw that at least partially covers the distal end face. In this way, the tissue can be fixed between the first surface and the second surface in the closed position. Further, tissued can be fixed between the distal end face and a surface area of the second jaw that at least partially covers the distal end face. The first surface and the second surface are the faces of the first jaw and the second jaw, respectively, that face each other in the open and/or closed position. In the closed position, the first surface may extend parallel to the second surface.

The pair of jaws may be pivotable relative to each other about the joint axis that lies transverse to a longitudinal axis of the coaxial transmission line. In one example, the pair of jaws comprises a static jaw that is fixed relative to the instrument shaft, and a movable jaw that is pivotably mounted relative to the static jaw to open and close the gap between the opposing inner surfaces. The energy delivery structure may be disposed on the inner surface of the static jaw. In another example, both jaws are arranged to pivot with respect to the instrument shaft, e.g. in a symmetrical forceps-type or scissors-type arrangement. Relative movement of the pair of jaws may be controlled from a handle at a proximal end of the instrument shaft. A control rod or control wires may pass through the instrument shaft to operably couple an actuation mechanism on the handle to the pair of jaws.

In another example, the pair of jaws may be arranged to move relative to one another in a manner that maintains the inner surfaces thereof in an aligned, e.g. parallel, orientation. This configuration may be desirable for maintaining a uniform pressure on grasped tissue along the length of the jaws. One example of such a closure mechanism is disclosed in WO 2015/097472.

The first jaw and/or the second jaw may have a Maryland configuration. This can include that the first jaw and the second jaw are not straight but bent/curved, e.g. forming an arc or an S-shape in a side view. Of course, the first jaw and/or the second jaw may have a straight configuration.

The first surface includes some exposed sections of the first electrode and the second electrode. Other sections of the first electrode and the second electrode are exposed on the distal end face. The first electrode and/or the second electrode are arranged within and/or on the first jaw. The first electrode and/or the second electrode are made from an electrically conductive material, such as metal, and may be connected to an inner conductor and an outer conductor of the coaxial cable, respectively.

The first electrode and the second electrode are electrically isolated from each other by the first isolating portion. Further, the sections of the first electrode and the second electrode that are exposed on the first surface and/or the distal end face are spaced apart from each other, for example by an air gap or the first isolating portion (e.g. an exposed section thereof).

The first surface and the distal end face may form a continuous surface of the first jaw. The first surface and the distal end face may be inclined relative to each other. For example, an angle between a plane defined by the first surface and a plane defined by the distal end face may form an angle between 10° to 90°, optionally 45°, 60°, or 90°. The distal end face, the first surface, and an outer surface of the second electrode may define an outer surface of the first jaw. In this configuration, the distal end face is a side surface while the first surface and the outer surface of the second electrode are surfaces that extend along the longitudinal direction of the first jaw. In other words, the distal end face extends transverse to the longitudinal direction of the first jaw. The distal end face may be a surface of the first jaw that is arranged at a distalmost position of the first jaw. In other words, when moving the first jaw along the longitudinal direction of the first jaw towards the tissue, the distal end face firstly and/or solely contacts the tissue.

The distal end face may be straight/flat. Alternatively, the distal end face may be curved. The sections of the first electrode, the second electrode, and the first isolating portion that are exposed at the distal end face may be flush with respect to each other or define a flat surface. Alternatively, the sections of the first electrode and/or the second electrode that are exposed at the distal end face may protrude from the section of the second isolating portion that is exposed at the distal end face.

The second jaw may partially or completely cover the first surface and a distal end face in the closed position of the first jaw and the second jaw. In any case, the second jaw covers the section of the first electrode that is exposed at the distal end face and/or the section of the second electrode that is exposed to the distal end face in a closed position. Stated differently, if the first jaw and the second jaw are brought together with no tissue therebetween, the second jaw covers and/or contacts the sections of the first electrode and/or the second electrode that are exposed on the first surface and/or the distal end face. In this way, the second jaw may function to shield the first and/or second electrode(s) when the jaws are closed, for example, to avoid unintentionally treating tissue whilst the instrument is moved into position at a treatment site.

The sections of the first electrode and the second electrode exposed at a distal end face can define an active electrode and a return electrode, respectively, which can be used for cutting tissue at the distal end face. The first electrode and the second electrode can be connected to the transmission line which is configured to convey both microwave and radiofrequency energy.

In an optional embodiment, the second jaw includes an overhang portion protruding from the second surface. Optionally the overhang portion covers sections of the first electrode and/or the second electrode that are exposed on the distal end face in the closed position of the first jaw and the second jaw.

The overhang portion may include a side surface which is continuous with the second surface. The second surface and the side surface may be inclined to each other. For example, an angle between a plane defined by the second surface and a plane defined by the side surface may form an angle between 10° to 90°, optionally 45°, 60°, or 90°. This angle may correspond to (e. g. be identical to) the angle defined by the first surface and the distal end face. In the closed position, the distal end face and side surface may extend parallel to each other and/or contact each other.

The overhang portion of the second jaw may provide the first and second jaws with a shape similar to a hooked-shaped beak of a bird of prey. In the longitudinal direction of the first jaw and the second jaw (e.g. measured from the pivot axle), the second jaw may have a greater length along the longitudinal direction compared to the first jaw in the closed position. The difference in length between the first jaw and the second jaw can correspond to the length of the overhang portion along the longitudinal direction. In other words, without the overhang portion, the first jaw and the second jaw may have the same length and/or configuration. The overhang portion protrudes from the second surface towards the first jaw and partially or completely covers the distal end face.

Tissue can be clamped or grasped between the distal end face and the side surface of the overhang portion. The surface areas of the side surface and the distal end face are significantly smaller than the surface areas of the first surface and the second surface. Therefore, smaller portions of tissue can be grasped between the distal end face and the side surface compared to clamping tissue between first surface and the second surface. This allows finer cutting of tissue that is clamped between the distal end face and the side surface.

In an optional embodiment, sections of the first and second electrodes that are exposed on the first surface define a first sealing area and a second sealing area on the first surface. Further optionally, the first sealing area is spaced from the second sealing area to form a gap therebetween on the first surface so that the tissue between the first sealing area and the second sealing area is not sealed upon the emission of the microwave electromagnetic energy. Further optionally, the first and second sealing areas are configured to seal the tissue on either side of the gap using the emitted microwave electromagnetic energy.

The exposed sections of the first electrode and/or the second electrode on the first surface may extend as lines and/or form areas on the first surface. In particular, the exposed sections of the first electrode and the second electrode on the first surface define two sealing areas laterally offset on the first surface, namely the first sealing area and the second sealing area. The first sealing area and the second sealing area may be connected to each other on the first surface by the other parts of the exposed sections of the first electrode and the second electrode, e.g. the sections of the first electrode and the second electrode that are exposed on the distal end face. This means that the first sealing area and the second sealing area may or may not be connected to each other on the first surface and/or the distal end face. The sections of the first electrode and the second electrode that are exposed on the distal end face may also be spaced away from each other so that the exposed sections of the first electrode and the second electrode are not connected to each other over the complete first surface and distal end face.

In any case, the first sealing area and the second sealing area are separated by a gap on the first surface. For example, the first sealing area includes a first set of exposed sections of the first electrode and the second electrode which extends alongside (e.g. and spaced from, in a side-by-side manner) the second sealing area, wherein the second sealing area includes a second set of exposed sections of the first electrode and the second electrode. For example, the first sealing area and/or the second sealing area define elongated structures (e.g. rectangles or ellipses) that are arranged side-by-side and separated by the gap on the first surface.

The exposed sections of the first electrode on the first surface may form (straight) lines which are separated by the gap. The exposed sections of the second electrode on the first surface may form (straight) lines between which the exposed sections of the first electrode are positioned. Exposed sections of the first isolating portion may be arranged between the (straight) lines of exposed sections of the first electrode and the second electrode on the first surface.

The first sealing area and the second sealing area are each configured to emit microwave energy at the exposed sections of the first electrode and the second electrode. For example, the exposed section of the first electrode may be considered a dipole antenna for radiating microwave electromagnetic energy. This applies for both the first sealing area and the second sealing area. This means that both the first sealing area and the second sealing area include two electrodes of different polarity or electrical potential.

The first sealing area and the second sealing area are configured to emit microwave energy to tissue that is close to or in contact with the exposed sections of the first electrode and the second electrode. The exposed sections of the first electrode and the second electrode are arranged such that tissue that is clamped and/or grasped between the first surface and the second surface in the closed position is in contact with the exposed sections of the first electrode and the second electrode or in contact with the first sealing area and the second sealing area. This means that the first sealing area and the second sealing area have a double functionality. They can clamp or grasp tissue (with the second surface being the counterpart) and emit microwave energy.

The greatest intensity of the emitted microwave energy is achieved in a portion of the tissue that is in contact with or directly above the exposed sections of the first electrode and the second electrode in the first sealing area and the second sealing area. In particular, the intensity of the emitted microwave energy is highest at respective edges or corners of the exposed sections of the first electrode and the second electrode that face each other. In other words, the intensity of the emitted microwave energy is highest above the interface of the first isolating portion and the first electrode (at the first surface) and at the interface of the first isolating portion and the second electrode (at the first surface). Thus, portions of the exposed sections of the first electrode that are away from the respective exposed sections of the second electrode exhibit a (significantly) lower intensity of the emitted microwave energy. These portions may be regarded as not corresponding to the first and/or second sealing areas since, due to the reduced intensity, no or insufficient microwave sealing can be achieved.

Further, (significantly) less microwave energy is absorbed by tissue that is arranged above or in contact with the area of the first surface that is arranged between the first sealing surface and the second sealing surface, i.e. in the area of the gap. This may mean that tissue is only sealed above the first sealing area and the second sealing area but tissue in an area between the first sealing area and the second sealing area is not sealed. In other words, if an intensity of the electromagnetic energy emitted directly at the exposed sections of the first electrode and the second electrode (i.e. at the first sealing area and the second sealing area) is set to 100%, the intensity of the electric energy measured in or above the gap (i.e. an area between the first sealing area and the second sealing area) is 60%, 50%, 40%, 30%, 25%, 20%, 10%, 5%, or less.

The intensity of the emitted microwave radiation is only sufficiently high to effect tissue sealing above the first sealing area and the second sealing area whereas the intensity of the emitted microwave energy is below a threshold for effecting tissue sealing in an area between the first sealing area and the second sealing area. The threshold for effecting tissue sealing depends on the tissue. However, due to the separation of the first sealing area and the second sealing area, an area between the first sealing area and the second sealing area can be established in which no or little (e.g. only negligible) tissue sealing is effected upon the emission of microwave energy. In other words, a dimension of the gap is chosen such that effective tissue sealing can be achieved over the first sealing area and the second sealing area but not in an area therebetween.

In an optional embodiment, the electrosurgical instrument further includes a cutting device for cutting tissue in the closed position. Optionally, the cutting device is effective along a cutting line on the first surface to cut the tissue in the closed position, the cutting line being positioned on the gap.

The cutting device is configured to cut or divide tissue that is arranged between the first sealing area and the second sealing area. The line of the cut or divide of the tissue may be considered the cutting line. The cutting device may be arranged on or can be moved along the cutting line. The cutting line is arranged between the first sealing area and the second sealing area, i.e. within the gap. Optionally, the cutting line is arranged in the middle between the first sealing area and the second sealing area so that a distance from the cutting line to the first sealing area is the same as a distance from the cutting line to the second sealing area.

The cutting device is therefore configured to cut tissue that is not sealed. For example, the tissue is firstly sealed and then cut. Due to the arrangement of the cutting device in the gap, the cutting device cuts in a portion of the tissue that is not sealed although other portions of the tissue have been sealed due to the emission of microwave energy. This means that sealing and cutting of the tissue may be simultaneously conducted because little or no interaction between the sealing and the cutting is expected due to the spatial separation of the sealing and the cutting.

In an optional embodiment, the second electrode covers the first electrode on the side of the first electrode facing away from the first surface and/or the first isolating portion is arranged between the first electrode and the second electrode.

The second electrode may completely cover the first electrode on one side of the first surface so that the second electrode shields the first electrode. The second electrode may act and/or form a half-shell or channel in which the first electrode is arranged. For example, the second electrode may be a ground electrode. In the closed position, the second electrode is configured to shield microwave radiation that is emitted from the first electrode away from the first surface. Thus, the first electrode may solely be configured to emit microwave energy through or on the first surface.

In an optional embodiment, portions of the first electrode and/or the second electrode are plate-shaped and respectively include end faces, wherein optionally the end faces form the exposed sections. Optionally, in a cross-sectional view of the first jaw, the portions of the first electrode and/or the second electrode are U-shaped or V-shaped.

For example, the portions of the first electrode and/or the second electrode that extend along sections of the first jaw where the first and second sealing areas are provided may be plate-shaped. Other portions of the first electrode and/or the second electrode may have different configurations. For example, proximal and/or distal end portions of the first electrode and/or the second electrode may have shapes that deviate from a plate shape. This may be provided for forming distal and/or proximal end portions of the jaw. End faces of the plate-shaped portions of the first electrode and/or the second electrode can be in the exposed sections on the first surface.

The plate-shaped portions of the first electrode and/or the second electrode may have a shape of the letter U, V, or variations thereof in a cross-sectional view of the first jaw. For example, the first electrode may be arranged within the shape defined by the U-shape or V-shape in a cross-sectional view.

In an optional embodiment, the sections of the first electrode and the second electrode that are exposed on the first surface at least partially extend parallel to each other and/or each include two straight portions that extends parallel to each other.

The exposed sections of the first electrode and the second electrode completely or partially extend parallel to each other on the first surface. For example, two portions of the exposed sections are straight and are connected by a connecting section which may be exposed on the distal end face. The sections of the first electrode exposed on the first surface may completely or partially extend parallel to the exposed section of the second electrode. The straight portions of the exposed sections may form the first sealing area and the second sealing area. So, the distance between straight portions of the exposed sections defines the width of the gap. In the presence of the connecting section, the first electrode and/or the second electrode each form a single section that is exposed on the first surface.

In an optional embodiment, in the closed position, the cutting line is arranged between the parallel extensions and/or the straight portions of the sections of the first electrode that are exposed on the first surface.

The cutting line may extend parallel between the straight portions of the sections of the first electrode and/or the second electrode exposed on the first surface. In this case, the cutting line is straight. On the first surface, the exposed sections of the first electrode and the exposed sections of the second electrode partially surround the cutting line.

In an optional embodiment, sections of the first electrode that are exposed on a distal end face are spaced from sections of the second electrode that are exposed on the distal end face and/or from each other.

This means that two or more sections of the first electrodes that are exposed on the distal end face are spaced from each other. Further, two or more sections of the second electrode that are exposed on the distal end face are spaced from each other. The two or more sections of the first electrode that are exposed on the distal end face can each be spaced from the two or more sections of the second electrode that are exposed on the distal end face. There may be two or more pairs of sections of the first electrode and second electrode that are spaced from each other and extend alongside each other. Each respective pair may act as an active and return electrode for radiofrequency cutting at the distal end face. It is to be noted that the sections of the first electrode that are spaced apart on the distal end face are connected to each other underneath the distal end face. Likewise, it is to be noted that the sections of the second electrode that are spaced apart on the distal end face are connected to each other underneath the distal end face.

In this configuration, an exposed section of the first electrode on the first/second sealing area may be continuous with a respective exposed section of the first electrode on the distal end face. Similarly, an exposed section of the second electrode on the first/second sealing area may be continuous with a respective exposed section of the second electrode on the distal end face.

The first electrode may have two side walls and a bottom wall connecting the two side walls. The cavity defined by the two side walls and the bottom wall may be filled with the second isolating portion. The two side walls may be directly connected to the bottom wall and/or each wall may have a plate shape so that the first electrode may have a U-shape in a cross-sectional view of the first jaw.

The two side walls may be longer than the bottom wall along the longitudinal direction of the first jaw so that the two side walls distally protrude from the bottom wall. Distal faces of the side walls may form the sections of the first electrode that are exposed on the distal end face and/or top faces of the side walls may form the exposed sections of the first electrode on the first surface.

The first isolating portion may surround an outer surface of the two side walls and the bottom wall. Optionally, the first isolating portion may also be arranged between the sections of the side walls that distally extend beyond the bottom wall. In this embodiment, on the distal end face, the first isolating portion is exposed between the exposed sections of the first electrode and/or between the exposed sections of the first electrode and the second electrode. So, on the distal end face, only the first isolating portion can be exposed.

In an optional embodiment, the distal end face includes a single section of the first electrode that it exposed on the distal end face and a single section of the second electrode that is exposed on the distal end face.

In an optional embodiment, the section of the first electrode exposed on the distal end face and/or the section of the second electrode exposed on the distal end face form a connecting section connecting the first sealing area to the second sealing area.

In this embodiment, there may be only one section of the first electrode that is exposed on the distal end face and/or there may be only one section of the second electrode that is exposed on the distal end face. These sections may extend parallel to each other and/or are curved on the distal end face. For example, the sections of the first and second electrodes exposed on the distal end face may form a loop, arc, curve or half-circle which correspond to the connecting section. The connecting section may further include the exposed section of the first isolating portion which may extend between the exposed sections of the first electrode and the second electrode on the distal end face.

So, in this embodiment, there are continuous sections of the first electrode and the second electrode that are exposed on the distal end face and which provide the radiofrequency cutting. Stated differently, the section of the first electrode that is exposed on the first surface and the distal end face is a continuous electrode, which can extend over the first sealing area, the distal end face, and the second sealing area. Similarly, the section of the second electrode that is exposed on the first surface and the distal end face is a continuous electrode, which can extend over the first sealing area, the distal end face, and the second sealing area.

The side walls and the bottom wall of the first electrode may have in this embodiment approximately the same length. Distal faces of the side walls and the bottom wall may form the exposed sections of the first electrode on the distal end face. The exposure of the bottom wall may correspond to the connecting section.

In an optional embodiment, the first sealing area and the second sealing area define an angle of less than 180°.

This means that the first sealing area and the second sealing area do not define a flat area (not considering the cutting device and the exposed second isolating portion).

For example, the area of the first surface on one side of the cutting device is inclined to an area of the first surface on the other side of the cutting device. In another example, the exposed section of the second isolating portion provides a flat surface, while the first and second sealing areas define an angle of less than 180° with the flat exposed section of the second isolating portion. In this case, only the first sealing area and the second sealing area are inclined.

The exposed section of the second isolation portion may be flat and/or define an angle with the first sealing area and the second sealing area. Alternatively, the exposed section of the second isolation portion may include two surface areas, one of which forms a flat plane with the first sealing area and the other of which forms a flat plane with the second sealing area. The cutting line may be arranged at that line where the two surface areas of the exposed section of the second isolation portion meet. Further, the two surface areas of the exposed section of the second isolation portion may be symmetrical to the cutting line and/or each define an angle with the respective first and second sealing areas.

In an optional embodiment, the electrosurgical instrument further comprises a second isolating portion on the first jaw. Optionally, the second isolating portion is positioned to electrically isolate the cutting device from the first electrode.

The first isolating portion and/or the second isolating portion may be made from an electrically non-conductive material such as a ceramic (e.g. Zirconia or Alumina) or plastic material (e.g. Polyetheretherketon (PEEK) or Polytetrafluoroethylene (PTFE)). The first isolating portion and/or the second isolation portion may be fixed to the first electrode and/or the second electrode and/or vice versa. The first electrode may be sandwiched between the first isolating portion and the second isolating portion. For example, the second isolating portion may cover the side of the first electrode that faces the first surface except for the exposed sections of the first electrode. As an example, the second isolating portion is arranged in the space formed by the U-shaped or V-shaped portions of the first electrode.

In an optional embodiment, the cutting device includes a blade, wherein the second isolating portion includes a groove in which the blade is movable.

The blade may form a radiofrequency electrode and is electrically isolated from the first electrode by the second isolating portion. Here, the second isolating portion includes a groove along which the blade is movable. The groove defines the cutting line. The blade can be connected to radiofrequency energy so that the blade can provide radiofrequency cutting and/or mechanical cutting.

A third isolating portion arranged on the second jaw (described further below) may also include a groove which matches the groove in the second isolating portion so that the blade can be simultaneously moved in both grooves. In this embodiment, the second jaw does not include an active electrode for emitting microwave energy.

Alternatively, the second jaw includes the third electrode and a fourth electrode for emitting microwave energy. The radiofrequency electrode (e.g. the blade) may be an additional (fifth) electrode. In this case, the third isolating portion may include a groove which matches the groove in the second isolating portion so that the blade can be moved in both grooves similar to the above-described grooves.

The blade may be an active electrode, wherein one or more of the first to fourth electrodes may act as a return electrode.

The blade may be arranged on the second isolating portion or within the groove in the second isolating portion. The blade may be exposed at the first surface, e.g. the second isolating portion. The cutting line therefore extends over the second isolation portion. A width of the second isolating portion along the first surface may correspond to the width of the gap. The second isolating portion may delimit the first sealing area and/or the second sealing area. Optionally, a section of the second isolating portion that is exposed on the first surface is arranged between the first sealing area and the second sealing area. The section of the second isolating portion that is exposed on the first surface may correspond to the gap. The cutting line may be in the middle of the section of the second isolating portion that is exposed on the first surface. The blade may be actuated by an actuation rod or control wire that can extend within the instrument shaft and is connected to the handle. The blade may have a cutting edge which is sufficiently sharp to mechanically cut tissue which is clamped between the first jaw in the second jaw in the closed position.

In one example, the blade may be slidable in a longitudinal direction between a retracted position in which it lies proximal to the pair of jaws and an extended position in which it lies within the region between the pair of jaws. It is desirable for the blade to slide into the region between the blade when they are in the closed position. The blade may be slidable along a longitudinally extending recessed groove formed in the pair of jaws, i.e. in each of the first jaw and the second jaw, so that it can contact tissue held in the gap when the pair of jaws are closed. The groove may be arranged to act as a guide rail for the cutting blade, which may be particular useful where the pair of jaws curve towards their distal ends (e.g. having a Maryland configuration).

In another example, the blade may be mounted within one of the pair of jaws and may be slidable or otherwise movable in a lateral direction between a retracted position in which it lies beneath the inner surface of the jaw and an extended position in which it lies within the region between the pair of jaws.

The blade may comprise a rigid element with a sharp edge adapted to slice biological tissue, e.g. a scalpel-type blade or the like. This type of blade is configured to perform a “cold” cut, which may be preferred because it carries a low risk of collateral thermal damage that is associated with other cutting techniques. However, the invention need not be limited to a cold cut blade. In other examples, the blade may comprise any one of: a bipolar radiofrequency cutting element, an ultrasound sonotrode, and a heatable wire element.

It has been found that radiofrequency cutting can be less effective on tissue that has previously been sealed (by microwave energy or other means of tissue sealing).

In an optional embodiment, the electrosurgical instrument comprises a radiofrequency electrode which includes a ridge protruding from the second isolating portion.

In this case, the radiofrequency electrode may be a bar or rod made from an electrically conductive material (e.g. metal or metal alloy) that is arranged on or embedded in a recess of the second isolating portion. The radiofrequency electrode can be permanently fixed to the second isolating portion. The radiofrequency electrode may be arranged on the second isolating portion so that tissue can contact the radiofrequency electrode, especially in the closed position.

In an optional embodiment, the cutting device includes a radiofrequency electrode exposed on the second surface. Further optionally, the electrosurgical instrument comprises the second isolating portion arranged on the first jaw so that, in a closed position, the second isolating portion electrically isolates the radiofrequency electrode from the first electrode, wherein optionally the second isolating portion is made from silicone or a silicone rubber.

In this case, the radiofrequency electrode is arranged on the second jaw. For example, the radiofrequency electrode may be configured to be in contact or close to the second isolating portion in the closed position. The radiofrequency electrode may be electrically isolated from other parts of the second jaw, for example by a third isolating portion. The arrangement of the radiofrequency electrode on the third isolating portion may include the same configuration, features, and/or characteristics as the arrangement of the radiofrequency electrode on the second isolating portion described above.

Silicone is more flexible and softer compared to other types of electrically isolating material. This may reduce the pressure applied to the tissue between the radiofrequency electrode and the second isolating portion in the closed position. It has been found that RF cutting is more effective if less pressure is applied during microwave sealing. The reduction of pressure may be achieved by the provision of the second isolating portion made from silicone or silicone rubber.

In an optional embodiment, the electrosurgical instrument includes a third electrode for emitting microwave energy which is exposed on the second surface and/or a fourth electrode for emitting microwave energy which it is exposed on the second surface.

The first electrode and/or the second electrode may be symmetrical to the third electrode and/or fourth electrode, respectively—both relative to the first and second surface in the closed position. More generally, the characteristics, features and preferred embodiments described in conjunction with the first and/or second electrodes may equally apply for the third and/or fourth electrodes, respectively. The third electrode may be electrically isolated from the fourth electrode by the fourth isolating portion. The third electrode may be covered by the third isolating portion.

In an optional embodiment, the first electrode is electrically connected to the third electrode and/or the second electrode is electrically connected to the fourth electrode.

For example, the first electrode and third electrode are electrically connected to an inner conductor of the coaxial cable while the second electrode and the fourth electrode are connected to the outer conductor of the coaxial cable. There may be a direct electrical connection between the first electrode and third electrode and/or between the second electrode and fourth electrode. Alternatively, the electrical connection is provided by the connection to the same conductors of the (coaxial) transmission line.

This arrangement allows the application of microwave energy to the tissue from two sides. If the first jaw and the second jaw are symmetrically configured, this allows a symmetrical application of microwave energy from the top and bottom after tissue being clamped in the closed position. Further, the paired configuration of the first and third electrodes as well as the second and fourth electrodes (whereby the first and third electrodes can be the inner electrodes, i.e. respectively surrounded by the second and fourth electrodes) can create a more concentrated electromagnetic/microwave field increasing the efficiency of the emitted electromagnetic energy for sealing and/or cutting.

In an optional embodiment, the electrosurgical instrument further comprises a first coaxial cable, which is connected to the first electrode and the second electrode, and a second coaxial cable which is connected to the third electrode and the fourth electrode.

It has been observed that the emission distribution of the microwave energy varies along the extension of the exposed sections of the first electrode and the second electrode. This equally applies to the exposed sections of the third electrode and the fourth electrode. The emission distribution is frequency-dependent. To provide a more uniform distribution of the microwave energy into the tissue, the first and second electrodes may receive microwave energy of a first frequency while the third and fourth electrodes may receive microwave energy of a second frequency. Due to the difference in frequency, the emission distribution of the first and second electrodes differs from the emission distribution of the third and fourth electrodes. This may result in a local minimum of the emitted microwave energy at the first jaw being aligned with a local maximum of emitted microwave energy at a second jaw (in the closed position). This may be if the first coaxial cable is connected to a first generator generating a first frequency while the second coaxial cable is connected to a second generator generating the second frequency. The same or a similar tissue effect of evening the heating/sealing using multiple frequencies can be achieved in a first and second configuration as well as third and fourth configuration by combining frequencies at the generator and using multi frequency antenna geometry.

In an optional embodiment, the groove is open at the distal end face so that the blade can be moved beyond the distal end face in the open position.

The groove may have an open end, extend until the distal end face, and/or not be closed at the distal end face. This allows the blade to move along the groove such that a cutting edge of the blade can be arranged/positioned to be exposed on the distal end face or protrude from the distal end face. Stated differently, the blade can be used to cut tissue that is in contact with the distal end face. For example, tissue may be grasped or clamped between the distal end face and the side surface of the overhang portion which then can be cut or divided by advancing the blade beyond the groove/distal end face.

In an optional embodiment, the second isolating portion is exposed on the distal end face and the groove in the second isolating portion extends until the distal end face.

This means that the groove is open at the distal end face allowing that the blade can be advanced beyond the distal end face. Further, the second isolating portion provides the groove and extends until the distal end face. The section of the second isolating portion that is exposed on the distal end face may be arranged between the sections of the first electrode that are exposed on the distal end face. So, the blade is electrically isolated from the first electrode on the distal end face.

In an optional embodiment, the groove ends before the distal end face.

In this embodiment, a portion of the first isolating portion and/or the second isolating portion may provide an end of the groove against which the blade abuts if it is maximally moved along the groove in a distal direction. In this embodiment, the blade cannot be used for cutting tissue that is clamped or grasped between the distal end face and the side surface of the overhang portion.

In an optional embodiment, the electrosurgical instrument further comprises a fluid feed configured to convey a fluid to the distal end face.

The fluid may include a liquid, e.g. a saline and/or pure water, or gas for cooling the tissue at the distal end face. Radiofrequency cutting is usually associated with the generation of substantial amounts of heat which can be cooled by the fluid that is conveyed to the distal end face.

The fluid feed may include one or more pipes and/or hoses that extend through the first jaw and/or the second jaw, for example through the first, second, and/or third isolating portions.

The fluid feed may provide a drip feed. Such a fine saline/fluid feed could be particularly useful in liver resection applications used in conjunction with any cutting at the distal end face, e. g. using a blade (“cold cut”) and/or using radiofrequency cutting.

In an optional embodiment, the fluid feed comprises at least one port (or outlet or nozzle) for outputting the fluid. Optionally, the port is arranged on the distal end face and/or on the side surface of the overhang portion contacting the distal end face in the closed position.

One or more ports are provided which may be implemented by ends of the pipes and/or hoses which convey the fluid. Alternatively, the ports are openings in the distal end face and/or the side surface which are connected to the fluid feed. Stated differently, the ports are outlets for the fluid so that the fluid can be supplied to the tissue that is grasped between the side surface and distal end face.

In an optional embodiment, the second jaw includes a third isolating portion which is exposed on the second surface and/or the overhang portion. Optionally, only the third isolating portion is in direct contact with the first jaw in the closed position.

The third isolating portion can be made from the same materials as the first isolating portion and/or the second isolating portion. The third isolating portion can be provided for electrically insulating the blade and/or the third electrode from the fourth electrode in the closed position.

The third isolating portion may be arranged so that the fourth electrode is exposed at the second surface and/or the distal end face. In this embodiment, the third isolating portion and the fourth electrode form the second surface. Alternatively, the third isolating portion is arranged so that only the third isolating portion is exposed on the second surface and/or the distal end face. In this case, the third isolating portion completely covers the fourth electrode on the second surface and/or distal end face. In this embodiment, the pressure applied to the tissue in a closed position can be reduced or more evenly distributed due to the softness of flexibility of the third isolating portion which can be made from silicone or silicone-based material.

In an optional embodiment, the cutting device includes a radiofrequency electrode which optionally includes a ridge protruding from the second isolating portion.

In this case, the radiofrequency electrode may be a bar or rod made from an electrically conductive material (e.g. metal or metal alloy) that is arranged on or embedded in a recess of the second isolating portion. The radiofrequency electrode can be permanently fixed to the second isolating portion. The radiofrequency electrode may be arranged on the second isolating portion so that tissue can contact the radiofrequency electrode, especially in the closed position. The radiofrequency electrode may not extend until the distal end face. In other words, the radiofrequency electrode may not be exposed on the distal end face.

According to a second aspect of the present invention, there is provided an electrosurgical apparatus for sealing and cutting tissue which comprises a generator unit for generating radiofrequency and/or microwave electromagnetic energy and the electromagnetic electrosurgical instrument as described above. The transmission line is configured to convey the radiofrequency and/or microwave electromagnetic energy from the generator unit to the first electrode, the second electrode and/or the cutting device.

The generator unit may be configured to generate electromagnetic energy of a fixed single frequency or of a plurality of fixed single frequencies. Alternatively or additionally, the generator unit may be tunable to generate electromagnetic energy of various frequencies, for example in a continuous range of frequencies between a minimum frequency and a maximum frequency. The generator unit may be connected to a power supply which provides the energy for generating the radiofrequency electromagnetic energy and/or microwave electromagnetic energy.

The generator unit is electrically and/or electronically (directly or indirectly) connected to the transmission line. Optionally, the generator unit generates the radiofrequency energy and/or microwave energy which is conveyed by the transmission line to the first to fourth electrodes where the radiofrequency energy and/or microwave energy is radiated into the treatment zone.

In an optional embodiment, the generator unit is configured to simultaneously generate microwave electromagnetic energy of the first frequency and microwave electromagnetic energy of a second frequency.

For example, the generator unit includes a generator that is configured to simultaneously generate electromagnetic energy of two different (fixed) frequencies.

Alternatively, the generator unit includes a first generator for generating electromagnetic energy of the first frequency and a second generator for generating electromagnetic energy of the second frequency. The output of the first generator and output of the second generator can be combined using a multiplexer.

The multiplexer may be a diplexer and can combine the input from various sources into one output. For example, a multiplexer (or diplexer) is used to combine the output of first and second generators to a single output which is connected or coupled to the transmission line.

In an optional embodiment, the first coaxial cable is connected to the generator unit to receive the first frequency and the second coaxial cable is connected to the generated unit to receive the second frequency.

Here again, the generator unit may include a first generator for generating electromagnetic energy of the first frequency and a second generator for generating electromagnetic energy of the second frequency. The first coaxial cable is connected to the first generator and the second coaxial cable is connected to the second generator.

In an optional embodiment, the generator unit is configured to simultaneously or alternatingly generate microwave electromagnetic energy of the first frequency and radiofrequency electromagnetic energy of a third frequency.

The generator unit may include a first generator for generating electromagnetic energy of the first frequency, a second generator for generating electromagnetic energy of the second frequency, and/or a third generator for generating electromagnetic energy of the third frequency. The output of the first generator and the output of the second generator may be combined as described above using a multiplexer.

The output of the third generator may be combined with the output of the multiplexer using a combiner which can include a switch for alternatingly switching between outputting the output of the multiplexer and outputting the output of the third generator. The combiner may include an additional multiplexer for combining the output of the multiplexer and the output of the third generator to simultaneously emit electromagnetic energy of the first frequency, the second frequency, and the third frequency. In this case, microwave sealing and radiofrequency cutting can be simultaneously effected.

If switching between the output of microwave energy and radiofrequency energy is possible, this can be used for sealing the tissue is in microwave energy and then subsequently cutting the tissue using radiofrequency energy. Alternatively, the switch between the output of microwave energy and radiofrequency energy can be executed repeatedly and rapidly providing near simultaneous cutting and sealing.

### BRIEF DESCRIPTION OF THE DRAWINGS

Embodiments of the invention are described in detail below with reference to the accompanying drawings, in which:

FIG. 1 shows a schematic view of an embodiment of an electrosurgical apparatus;

FIG. 2 shows a perspective view of an embodiment of an electrosurgical instrument (in an open position) of the electrosurgical apparatus shown in FIG. 1;

FIG. 3 shows a perspective view of a first electrode of the electrosurgical instrument of FIG. 2;

FIG. 4 shows a perspective view of a further embodiment of an electrosurgical instrument (in an open position) of the electrosurgical apparatus shown in FIG. 1;

FIG. 5 shows side views of the electrosurgical instrument of FIG. 4 in a closed position (top image) and the open position (bottom image); and

FIG. 6 shows a cross-sectional perspective view of the electrosurgical instrument of FIG. 4 in the closed position.

### DETAILED DESCRIPTION; FURTHER OPTIONS AND PREFERENCES

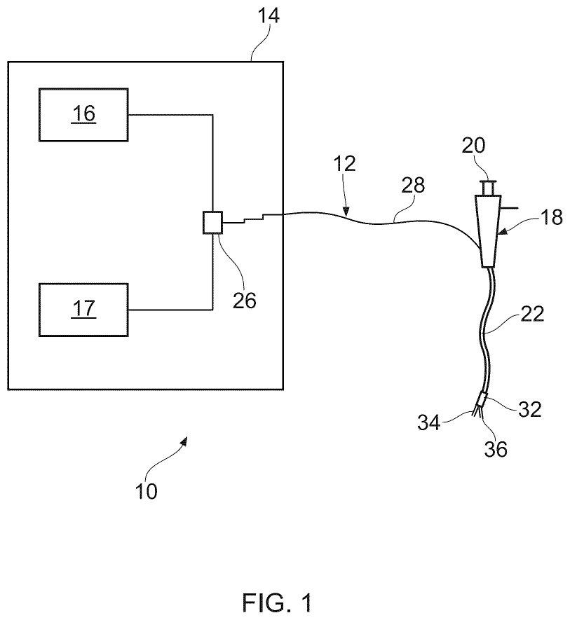

The present invention relates to an electrosurgical instrument and apparatus capable of delivering microwave energy to seal tissue (e.g. blood vessels) and/or of cutting the tissue. The electrosurgical instrument and apparatus may be used in open surgery but may find particular use in procedures where there is restricted access to the treatment site. For example, the electrosurgical instrument of the invention may be adapted to fit within the instrument channel of a surgical scoping device i.e. laparoscope, endoscope, or the like. FIG. 1 shows a schematic view of an electrosurgical apparatus 10 in which the electrosurgical instrument of the invention may be used.

FIG. 1 is a schematic diagram of an electrosurgical apparatus 10 that is an embodiment of the invention. The electrosurgical apparatus 10 is arranged to treat biological tissue using radiofrequency (RF) and/or microwave electromagnetic (EM) energy delivered from an electrosurgical instrument 12. The electromagnetic energy emitted by the electrosurgical instrument 12 into a treatment zone can be used to coagulate, cut, and/or ablate tissue in the treatment zone.

The electrosurgical apparatus 10 further comprises a generator unit 14 which can controllably supply radiofrequency and/or microwave electromagnetic energy to the electrosurgical instrument 12. The generator unit 14 may include a first generator 16 and a second generator 17. Suitable generators for this purpose are described in WO 2012/076844, which is incorporated herein by reference. The generator unit 14 may be arranged to monitor reflected signals received back from the electrosurgical instrument 12 in order to determine an appropriate power level for delivery. For example, the generator unit 14 may be arranged to calculate an impedance seen at the electrosurgical instrument 12 in order to determine an optimal delivery power level.

The electrosurgical apparatus 10 further comprises a surgical scoping device 18, such as a bronchoscope, endoscope, gastroscope, laparoscope or the like. The scoping device 18 may include a handpiece 20 and a flexible shaft 22. The handpiece 20 may include means for guiding the flexible shaft 22 through a cavity of a body. For example, the handpiece 20 can include means for moving a distal end of the flexible shaft 22 to change direction of the distal end of the flexible shaft 22. This helps maneuvering the flexible shaft 22 through the cavity of the body. The flexible shaft 22 may include a working channel through which elongated structures can be moved and, thus, positioned at the treatment zone within the cavity of the body.

The first generator 16 and the second generator 17 are each configured to generate electromagnetic energy of a fixed frequency. However, the generator unit 14 is not limited thereto; the first generator 16 and/or the second generator 17 can be configured to generate AC electromagnetic energy in a continuous range between a minimum frequency and a maximum frequency. The frequency of the electromagnetic energy to be generated by the first generator 16 and/or the second generator 17 may be selected using an interface (not shown in the figures).

The generator unit 14 can include a combiner 26 which is configured to temporally switch between outputting the output of the first generator 16 or the output of the second generator 17. The combiner 26 may also be configured to combine the outputs of the first generator 16 and of the second generator 17. In this case, the combiner 26 acts as a multiplexer or diplexer.

The generator unit 14 is thus capable of generating and controlling power to be delivered to the electrosurgical instrument 12, e.g. via a transmission line 28, which extends from the generator unit 14 through the surgical scoping device 18 and instrument channel to the distal tip of the instrument channel. The generator unit 14 may have a user interface for selecting and/or controlling the power delivered to the electrosurgical instrument 12, e.g. controlling the first and/or the second generators 16, 17 and/or the combiner 26. The generator unit 14 may have a display for showing the selected energy delivery mode. In some examples, the generator unit 14 may allow for an energy delivery mode to be selected based on the size of the vessel to be sealed. Alternatively or additionally, the energy delivery is adapted based on tissue state.

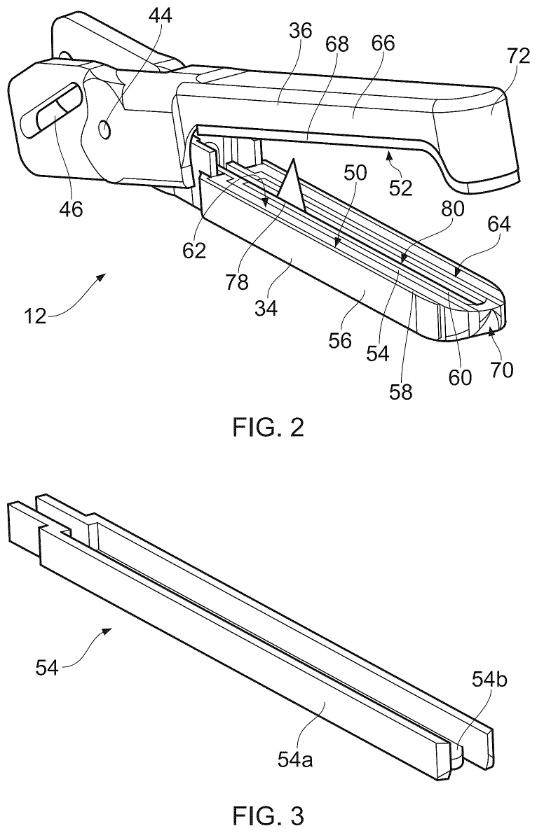

As shown in FIGS. 2 to 6, the electrosurgical instrument 12 can include the transmission line 28, an instrument shaft (not shown in the figures), a joint (not shown in the figures), a first jaw 34, and/or a second jaw 36 which will be discussed in further detail below. The transmission line 28 may include a single coaxial cable that connects the generator unit 14 to the first jaw 34 and/or second jaw 34 for conveying the radiofrequency and/or microwave energy (see FIG. 6).

The first jaw 34 and the second jaw 36 are operably coupled to the joint 32 that is mounted on a distal end of the instrument shaft. Both the first and second jaws 34, 36 may be arranged to pivot relative to the joint. The joint may be arranged to ensure that the jaws remain laterally aligned as they are moved together.

In an alternative embodiment, the pair of jaws 34, 36 comprises a static jaw that is fixed relative to the instrument shaft or the joint. The other jaw is pivotable or rotatable.

The joint includes a pivot axle 44 which defines a pivot axis. The first jaw 34 and the second jaw 36 can pivot around the pivot axis or pivot axle 44. For example, the pivot axle 44 is fixed to the joint and the first jaw 34 and the second jaw 36 can rotate around the pivot axle 44.

The joint may have a clevis structure and slots on the arms of the clevis structure. The slot may have an elongate shape and can be a through-hole or a recess in an inner surface of the clevis structure. The first jaw 34 and/or the second jaw 36 also include elongated slots 46 (see FIGS. 2 and 4 to 6). A control wire or actuation rod includes a cam (not shown) which is inserted in the slots of the clevis structure and in the slots 46 of the first jaw 34 and the second jaw 36. The engagement of the cam with the slots provides an actuation mechanism which translates a back-and-forth movement of the control wire (and thus of the cam) into a rotational movement of the first jaw 34 and the second jaw 36 around the pivot axle 44.

In use, the first jaw 34 and the second jaw 36 are intended to grip biological tissue (in particular a blood vessel) therebetween. The first jaw 34 and the second jaw 36 are arranged to apply pressure to the biological tissue between the opposed surfaces of the jaws 34, 36 and deliver energy (preferably microwave and/or radiofrequency electromagnetic energy) into the tissue from the transmission line 28.

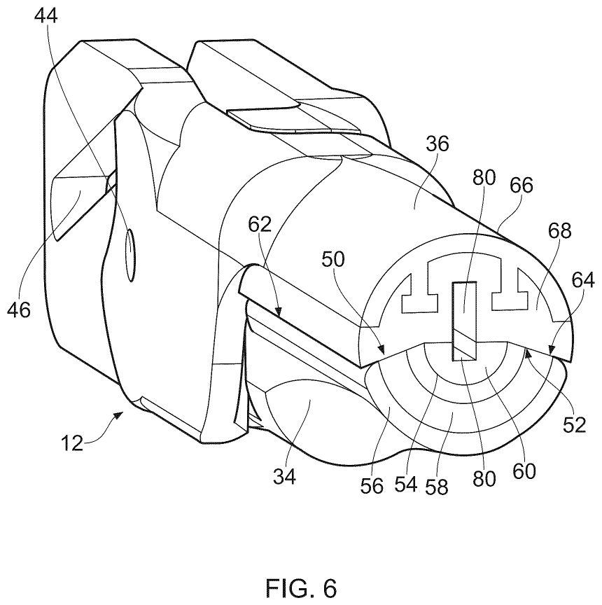

The first jaw 34 includes a first surface 50 which opposes a second surface 52 of the second jaw 36. The first surface 50 and/or the second surface 52 may form an outer surface of the first jaw 34 and the second jaw 36 respectively, which can be brought into contact with each other when the jaws 34, 36 are in the closed position (see FIG. 5). For example, the first surface 50 and the second surface 52 may be considered pressure pads or pressure areas with which pressure can be applied to the tissue grasped between the first jaw 34 and the second jaw 36.

In the embodiment of FIGS. 2 and 3, the first jaw 34 includes a first electrode 54, a second electrode 56, a first isolating portion 58, and/or a second isolating portion 60. The first electrode 54 and the second electrode 56 are made from an electrically conductive material, such as metal or a metal alloy. The second electrode 56 may form the outer surface of the first jaw 34 and/or may provide the connection to the pivot axle 44. Further, the slot 46 of the first jaw 34 may be provided in the second electrode 56. Thus, the second electrode 56 may have a function of providing the stability of the first jaw 34.

The second electrode 56 may have a form of a half-shell in a portion along the first surface 50. The second electrode 56 may surround the first electrode 54, the first isolating portion 58 and/or the second isolating portion 60. This means that the first electrode 54, the first isolating portion 58, and/or the second isolating portion 60 may be embedded in the half-shell of the second electrode 56. The shape of the second electrode 56 may also be considered as providing a recess or channel in which the first electrode 54, the first isolating portion 58 and/or the second isolating portion 60 are arranged.

The first isolating portion 58 electrically isolates the first electrode 54 from the second electrode 56. The first isolating portion 58 may be made from an electrically non-conductive material such as a ceramic material (e.g. including Zirconia), PEEK, silicone, and/or other plastic materials. The first isolating portion 58 may be sandwiched between the first electrode 54 and the second electrode 56.

The first electrode 54 and the second electrode 56 may have a U-shape or V-shape (see FIGS. 3 and 6) in a cross-sectional view along a section of the first jaw 34. Further, the first electrode 54 and the second electrode 56 may have a plate-shape (see FIGS. 3 and 6) along this section of the first jaw 34. At a distal end of the first jaw 34, the first electrode 54 and/or the second electrode 56 may have a different configuration so that the first electrode 54 and/or the second electrode 56 do not have an open end but a closed end. This means that the second electrode 56 can shield the first electrode 54 in all directions away from the first surface 50.

The first electrode 54 and/or the second electrode 56 are exposed at the first surface 50 to allow for contact with the tissue clamped between the first jaw 34 and the second jaw 36. In particular, two sections of the first electrode 54 and the second electrode 56 are exposed on the first surface 50 which are spaced from each other by a gap. In the embodiment shown, the two exposed sections are straight and extend in a direction of extension of the first jaw 34. Each exposed section includes an exposed section of the first electrode 54, the second electrode 56, and the first isolating portion 58. The two exposed sections provide a first sealing area 62 and a second sealing area 64 which are separated from each other by the gap in a direction perpendicular to the extension of the first jaw 34 (e.g. a longitudinal direction of the first jaw 34).

The first sealing area 62 and the second sealing area 64 are each provided for the emission of microwave energy so that tissue is sealed at the first sealing area 62 and the second sealing area 64. In particular, the intensity of the microwave energy that is emitted from the first electrode 54 and the second electrode 56 is highest at the sealing areas 62 and 64. The intensity of the emitted microwave energy is considerably lower in the area of the gap (i.e. between the first sealing area 62 and the second sealing area 64) compared to the first sealing area 62 and the second sealing area 64, for example 10% to 80% less than over the first sealing area 62 and the second sealing area 64. This means that tissue is effectively sealed at the first sealing area 62 and at the second sealing area 64 but not in the gap therebetween.

The first sealing area 62 and the second sealing area 64 may be provided by the end faces of the plate-shaped first electrode 54, the second electrode 56, and the first isolating portion 58 (see FIG. 3). The exposed sections of the first electrode 54 and the second electrode 56 over the first sealing area 62 and the second sealing area 64 may form two parallel straight lines which are respectively separated by the gap.

In the embodiment of FIGS. 2 and 3, the separation of the first sealing area 62 and the second sealing area 64 by the gap is provided by the second isolating portion 60. The second isolating portion 60 separates the exposed sections of the first electrode 54 on the first surface 50. In other words, the second isolating portion 60 separates the first sealing area 62 from the second sealing area 64. The second isolating portion 60 may be received in a cavity formed by the U-shaped first electrode 54. In other words, the first electrode 54 forms a channel similar to the second electrode 56. The second isolating portion 60 may be embedded or arranged in this channel.

As depicted in FIG. 3, the first electrode 54 may have two side walls 54a and a bottom wall 54b which connects the two side walls 54a. Each of the side walls 54a and the bottom wall 54b may have a plate shape. The side walls 54a and the bottom wall 54b may define a channel within which the second isolating portion 60 is arranged. The arrangement of the side walls 54a and the bottom wall 54b provides the U-shaped configuration in a cross-sectional view of the first electrode 54. The top end faces of the side walls 54a provide the exposed sections of the first electrode 54 on the first sealing area 62 and the second sealing area 64.

Along a longitudinal direction of the first jaw 34, the two side walls 54a are longer compared to the bottom wall 54b so that the two side walls 54a extend beyond the bottom wall 54b in the longitudinal direction of the first jaw 34. As such, side end faces of the side walls 54a may be exposed on a distal end face 70 of the first jaw 34 whereas the bottom wall 54b is not exposed at the distal end face 70.

The second isolating portion 60 may have a flat or straight outer surface at the first surface 50. For example, the exposed section of the second isolating portion 60 on the first surface 50 may be parallel to a lower surface of the second electrode 56 defining a lower surface of the first jaw 34 (see FIG. 6). The first sealing area 62 and the second sealing area 64 may be inclined relative to each other and relative to the exposed section of the second isolating portion 60. The angle between a plane defined by the exposed section of the second isolating portion 60 and a plane defined by the first and/or second sealing surface 62, 64 may be between 5° to 45°. Optionally, the first sealing area 62 and the second sealing area 64 define the same angle with the exposed section of the second isolating portion 60 at the first surface 50.

The inclined arrangement of the first sealing area 62 and the second sealing area 64 increases the respective surface areas without widening the first jaw 34. Further, after cutting the tissue clamped between the first jaw 34 and the second jaw 36, the cut tissue can be more easily released from the first jaw 34 compared to a flat (i.e. non-inclined) first surface 50.

The second isolating portion 60 may comprise a silicone-based material (e.g. silicone rubber) or silicone. Other examples include polyimide, PTFE or FEP type materials. These types of materials are more flexible or softer compared to a ceramic material which helps to reduce the pressure on the tissue over the area of the gap (i.e. between the first sealing area 62 and the second sealing area 64). So the pressure on the tissue can be reduced in an area where the tissue is not sealed. In other words, the first jaw 34 and the second jaw 36 press and seal the tissue at the first sealing area 62 and the second sealing area 64. In an embodiment, the first jaw 34 and the second jaw 36 press and seal the tissue only at the first sealing area 62 and the second sealing area 64.

The distal end face 70 is inclined to the first surface 50 by an angle of 90°. The distal end face 70 is a side surface of the first jaw 34 that comes into contact with tissue first if the first jaw 34 is advanced in the longitudinal direction of the first jaw 34 towards the tissue. In the embodiment of FIGS. 2 and 3, the distal end face 70 is curved. The first electrode 54 (e.g. the side walls 54a), the second electrode 56, and/or the first isolating portion 58 are exposed on the distal end face 70. The sections of the first electrode 54 that are exposed on the distal end face 70 are spaced away from each other. Similarly, the sections of the second electrode 56 that are exposed on the distal end face 70 are spaced from each other. Further, the sections of the first electrode that are exposed on the distal end face 70 are arranged between the sections of the second electrode 56 that are exposed on the distal end face 70. The sections of the first electrode 54 and the second electrode 56 that are exposed on the distal end face 70 are continuous with the respective sections of the first electrode 54 and the second electrode 56 that are exposed on the first surface 50. As such, the sections of the first electrode 54 and the second electrode 56 that are exposed on the distal end face 70 provide two pairs of electrodes similar to the first sealing area 62 and the second sealing area 64. Unlike the first sealing area 62 and the second sealing area 64, the two pair of electrodes on the distal end face 70 are provided for radiofrequency cutting. So, the first electrode 54 and the second electrode 56 are connected to the transmission line 28 which is configured to convey both microwave energy and radiofrequency energy e.g. includes a coaxial cable or a coaxial cable for transmitting/conveying microwave energy and wires for transmitting/conveying radiofrequency energy.

The surface area of the distal end face 70 is significantly smaller than the surface area of the first surface 50. Similarly, the sections of the first electrode 54 and the second electrode 56 that are exposed on the distal end face 70 are smaller than the first sealing area 62 and the second sealing area 64. This allows fine cutting using the exposed sections of the first electrode 54 and the second electrode 56 on the distal end face 70. For example, the exposed sections of the first electrode 54 and the second electrode 56 on a distal end face 70 may be used to cut holes into the tissue, e.g., as part of a tunnelling procedure.

The second jaw 36 includes a fourth electrode 66 and a third isolating portion 68. The fourth electrode 66 is made from an electrically conductive material, such as metal. The fourth electrode 66 may form the outer surface of the second jaw 36 and/or may provide the connection to the pivot axle 44. Further, the slot 46 of the second jaw 36 may be arranged on the fourth electrode 66. Thus, the fourth electrode 66 may have a function of providing the stability of the second jaw 36. The fourth electrode 66 may have a form of a half-shell in a portion of the second surface 52. The fourth electrode 66 may surround the third isolating portion 68. This means that the third isolating portion 68 may be embedded in the half-shell of the fourth electrode 66. The shape of the fourth electrode 66 may also be considered as providing a recess or channel in which the third isolating portion 68 is arranged.

The fourth electrode 66 may be mirror-symmetric to the second electrode 56. The fourth electrode 66 may have a U-shape (see FIG. 6) in a cross-sectional view along a section of the second jaw 36. Further, the fourth electrode 66 may have a plate-shape (see FIG. 6) along this section of the second jaw 36.

At a distal end of the second jaw 36, the fourth electrode 66 may have an overhang portion 72 which includes a side surface 74. The overhang portion 72 protrudes from the second surface 52 towards the first jaw on a distal end of the second jaw 36. The overhang portion 72 (completely) covers the distal end face 70 in the closed position. In particular, the overhang portion 72 covers the sections of the first electrode 54 and the second electrode 56 that are exposed at the distal end face 70. For example, the side surface 74 contacts the distal end face 70 in the closed position. As such, tissue can be clamped or grasped between the distal end face 70 and the side surface 74 and/or subsequently cut using radiofrequency energy emitted by the exposed sections of the first electrode 54 and the second electrode 56.

The overhang portion 72 may be a unitary component with the fourth electrode 66. Optionally, the overhang portion 72 includes an electrically conductive material that is in electrical connection with the fourth electrode 66. The configuration and arrangement of the fourth electrode 66 and/or the overhang portion 72 means that fourth electrode 66 and/or the overhang portion 72 can shield the first electrode 54 in directions away from the second surface 52 and/or the side surface 74.

The second electrode 56 and the fourth electrode 66 may be electrically connected to each other, either directly or by being connected to the same conductor of the transmission line 28. The first electrode 54 may be connected to an inner conductor of the coaxial cable and the second electrode 56 is connected to an outer conductor of the coaxial cable.

The third isolating portion 68 is made from an electrically non-conductive material, optionally a silicone-based material or silicone. The third isolating portion 68 may completely extend over the second surface 52 and the side surface 74. This means only the third isolating portion 68 contacts the first surface 50 and the distal end face 70 in the closed position.

Alternatively (not shown in the figures), the sections of the fourth electrode 66, of the overhang portion 72, and of the third isolating portion 68 that are exposed to contact tissue in the closed position may define or form the second surface 52. Thus, tissue can be grasped between the first electrode 54 and the second electrode 56 on the first jaw 34 and the fourth electrode 66 on the second jaw 36.

The electrosurgical instrument 12 may further include a cutting device 78. In the embodiment shown in FIGS. 2 to 6, the cutting device 78 includes a blade that is movable along a groove 80. The cutting device 78, e.g. the blade, may be mechanically coupled to an actuation rod or control wire with which the cutting device 78 can be moved along the groove 80.

The groove 80 is an example of a first groove or a second groove. The groove 80 is arranged within the second isolating portion 60 so that the cutting device 78 is electrically insulated from the first electrode 54. The groove 80 may be arranged to act as a guide rail for the cutting device 78. In the embodiment of FIG. 2, the groove 80 as defined by the second isolating portion 60 is open at a distal end so that the cutting device 78 abuts against the first isolating portion 58 that is arranged on the distal end face 70 between the exposed sections of the first electrode 54. Thus, in this embodiment, the cutting device 78 cannot be positioned so that it is exposed at the distal end face 70.

The third isolating portion 68 includes a further groove 80 which is aligned with the groove 80 in the second isolating portion 60 so that the cutting device 78 can move within the two grooves 80 in the closed position. This means the cutting device 78 is positioned in the grooves 80 in the closed position. In the open position, the cutting device 78 is only arranged in the groove 80 defined by the second isolating portion 60. As such, the groove 80 defined by the second isolating portion 60 may act as a guide rail for the cutting device 78 while the groove 80 defined by the third isolating portion 68 supports the cutting device 78 in the closed position.

The cutting device 78 may not be connected to the generator unit so that it is not configured to emit radiofrequency energy. The cutting device 78 may be considered providing a “cold cut”, i.e. only a mechanical cut. Alternatively, the cutting device 78 may be connected to the generator unit so that it is configured to emit radiofrequency energy. In this case, the cutting device 78 may cut tissue by radiofrequency cutting and/or mechanical cutting. The cutting device 78 is provided for cutting tissue that is grasped between the first jaw 34 and the second jaw 36, e.g. tissue that has been sealed before using microwave energy that was emitted by the first sealing area 62 and the second sealing area 64.

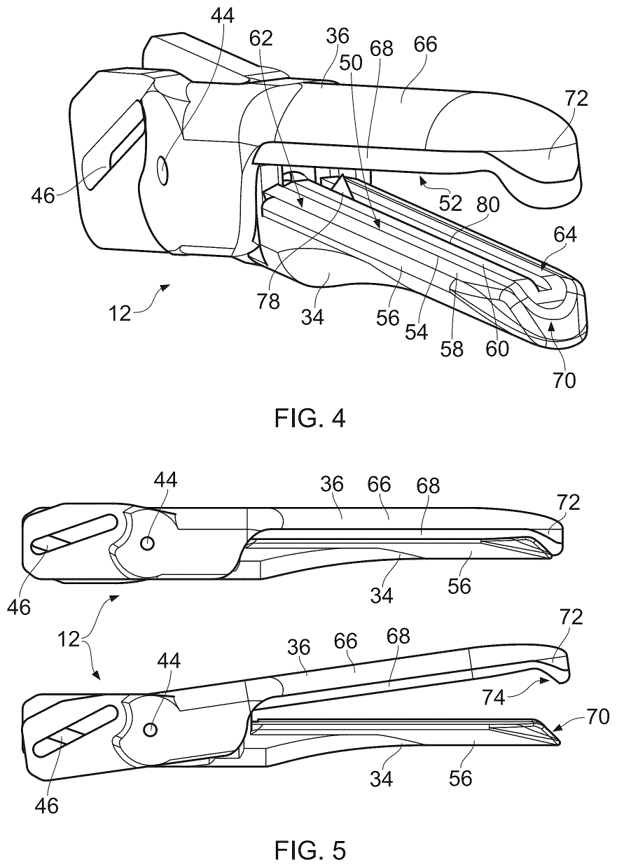

The embodiment of the electrosurgical instrument 12 shown in FIGS. 4 to 6 includes the same features, characteristics, and/or optional embodiments as the embodiment of the electrosurgical instrument 12 shown in FIGS. 2 and 3 except for the following differences.

The distal end face 70 is inclined to the first surface 50 by an angle of approximately 60° wherein the angle is defined between a plane defined by the first surface 50 and a plane defined by the distal end face 70. Similarly, the inclination of the side surface 74 to the second surface 52 is different to the inclination of the embodiment of FIGS. 2 and 3.

The distal end face 70 is straight, i.e. not curved as with the embodiment shown in FIGS. 2 and 3. There is only one section of the first electrode 54 that is exposed on the distal end face 70 and only one section of the second electrode 56 that is exposed at the distal end face 70. The exposed sections of the first electrode 54 and the second electrode 56 connect the first sealing area 62 to the second sealing area 64. The sections of the first electrode 54 and of the second electrode 56 that are exposed on the distal end face 70 form a half-circle, arc, curve or loop defining a connecting section which provides a continuous exposure of the first electrode 54 and the second electrode 56 over the first surface 50 and the distal end face 70.

The second isolating portion 60 extends until the distal end face 70 so that the second isolating portion 60 is exposed on the distal end face 70. The groove 80 defined by the second isolating portion 60 is open on the distal end face 70 so that the cutting device 78 can be advanced beyond the groove 80 defined by the second isolating portion 60. Consequently, the cutting device 78 can be exposed at the distal end face 70 for cutting tissue that is clamped or grasped between the distal end face 70 and the side surface 74. Further, the cutting device 78 in a position exposed at distal end face 70 can be used for cutting tissue in an open position, i.e. tissue that may be pressed against the distal end face 70.

1. An electrosurgical instrument for sealing or cutting tissue, comprising:
an instrument shaft comprising a transmission line for conveying microwave electromagnetic energy or radiofrequency electromagnetic energy;
a first jaw attached to the instrument shaft and including a first surface as well as a distal end face,
a second jaw attached to the instrument shaft and including a second surface,
a first electrode for emitting microwave electromagnetic energy or radiofrequency electromagnetic energy, and
a second electrode for emitting microwave electromagnetic energy or radiofrequency electromagnetic energy, and
a first isolating portion electrically isolating the first electrode from the second electrode,
wherein the first jaw and the second jaw can be moved between an open position, in which the tissue can be inserted between the first surface and the second surface, and a closed position, in which the first and second surfaces are brought together to clamp tissue therebetween,
wherein the first electrode and the second electrode are exposed on the first surface and on the distal end face, and
wherein, in the closed position, the second jaw covers the exposed first or second electrodes on the first surface and the distal end face.

2. The electrosurgical instrument according to claim 1, wherein the second jaw includes an overhang portion protruding from the second surface, wherein the overhang portion covers the exposed first or second electrodes on the distal end face in the closed position.

3. The electrosurgical instrument according to claim 1, further including a cutting device for cutting tissue in the closed position,
wherein sections of the first and second electrodes that are exposed on the first surface to define a first sealing area and a second sealing area on the first surface,
wherein the first sealing area is spaced from the second sealing area to form a gap therebetween on the first surface,
wherein the first and second sealing areas are configured to seal the tissue on either side of the gap using the emitted microwave electromagnetic energy, and
wherein the cutting device is effective along a cutting line on the first surface to cut the tissue in the closed position, the cutting line being positioned on the gap.

4. The electrosurgical instrument according to claim 1, wherein the second electrode covers the first electrode on a side of the first electrode facing away from the first surface or wherein the first isolating portion is arranged between the first electrode and the second electrode.

5. The electrosurgical instrument according to claim 3, wherein portions of the first electrode or the second electrode include end faces, wherein the end faces form the exposed sections, wherein, in a cross-sectional view of the first jaw, the portions of the first electrode or the second electrode are substantially U-shaped or V-shaped.

6. The electrosurgical instrument according to claim 3, wherein the sections of the first electrode and the second electrode that are exposed on the first surface at least partially extend parallel to each other, or
each include two straight portions that extend parallel to each other.

7. The electrosurgical instrument according to claim 6, wherein, in the closed position, the cutting line is arranged between the parallel extensions or the straight portions of the sections of the first electrode that are exposed on the first surface.

8. The electrosurgical instrument according to claim 1, wherein sections of the first electrode that are exposed on the distal end face are spaced from sections of the second electrode that are exposed on the distal end face or from each other.

9. The electrosurgical instrument according to claim 1, wherein the distal end face includes a single section of the first electrode that it exposed on the distal end face and a single section of the second electrode that is exposed on the distal end face.

10. The electrosurgical instrument according to claim 9, wherein the exposed section of the first electrode on the distal end face or the exposed section of the second electrode on the distal end face form a connecting section connecting a first sealing area to a second sealing area.

11. The electrosurgical instrument according to claim 3, wherein the first sealing area and the second sealing area define an angle of less than 180° with respect to each other.

12. The electrosurgical instrument according to claim 3, further comprising a second isolating portion on the first jaw, wherein the cutting device includes a blade, and wherein the second isolating portion includes a groove in which the blade is movable.

13. The electrosurgical instrument according to claim 12, wherein the groove is open at the distal end face so that the blade can be moved beyond the distal end face.

14. The electrosurgical instrument according to claim 13, wherein the second isolating portion is exposed on the distal end face and the groove in the second isolating portion extends until all the distal end face.

15. The electrosurgical instrument according to claim 12, wherein the groove ends before the distal end face.

16. The electrosurgical instrument according to claim 2, further comprising a fluid feed configured to convey a fluid to the distal end face.

17. The electrosurgical instrument according to claim 16, wherein the fluid feed comprises at least one port for outputting the fluid, wherein the port is arranged on the distal end face or a side surface of the overhang portion contacting the distal end face in the closed position.

18. The electrosurgical instrument according to claim 2, wherein the second jaw includes a third isolating portion which is exposed on the second surface or the overhang portion, wherein optionally only the third isolating portion is in direct contact with the first jaw in closed position.

19. An electrosurgical apparatus for sealing and cutting tissue, comprising:
a generator unit for generating radiofrequency or microwave electromagnetic energy, and
the electrosurgical instrument according claim 1,
wherein the transmission line is configured to convey the radiofrequency or microwave electromagnetic energy from the generator unit to the first electrode, the second electrode or a cutting device.

20. The electrosurgical apparatus according to claim 19, wherein the generator unit is configured to simultaneously or alternatingly generate microwave electromagnetic energy of a first frequency and radiofrequency electromagnetic energy of a third frequency.
